(19) United States
(12) Patent Application Publication
(10) Pub. No.: US 2021/0401368 A1
(43) Pub. Date: Dec. 30, 2021

(54) SYSTEM FOR TRANSDERMAL MONITORING OF ALCOHOL CONCENTRATION IN A TEST SUBJECT

(71) Applicant: Dräger Safety AG & Co. KGaA, Lübeck (DE)

(72) Inventors: Ingo NEUMANN, Lübeck (DE); Stefan MORLEY, Lübeck (DE); Martin ZIMMERMANN, Lübeck (DE)

(21) Appl. No.: 17/355,867

(22) Filed: Jun. 23, 2021

(30) Foreign Application Priority Data
Jun. 25, 2020 (DE) 10 2020 116 745.8

(51) Int. Cl. A61B 5/00

(52) U.S. Cl. CPC A61B 5/4845; A61B 5/0002; A61B 2560/0219; A61B 2560/0209; A61B 5/6828

(57) ABSTRACT

A system (100), for transdermal monitoring of the alcohol concentration (TAK) in a test subject (10), has an electronic measuring device (10) which is portable on the body of the test subject (1). A measuring module (11) transdermally detects measured values of the alcohol concentration (TAK) in the test subject (2). A transmission module (12) transmits the measured values to a relay station (20) having a receiving module (21), receiving measured values from the transmission module (12) and a sending module (22), sending received measured values to a remotely located monitoring station (30). An energy storage device (13, 23) provides an energy supply of the measuring module (11) which is uncoupled from the energy supply of the relay station (20).

### CROSS REFERENCE TO RELATED APPLICATIONS

This application claims the benefit of priority under 35 U.S.C. § 119 of German Application 10 2020 116 745.8, filed Jun. 25, 2020, the entire contents of which are incorporated herein by reference.

### TECHNICAL FIELD

The present invention pertains to a system for the transdermal monitoring of the concentration of alcohol in a test subject.

### TECHNICAL BACKGROUND

In many countries, for example, in the United States of America, there are programs, within the framework of which convicted criminals (convicts), who have committed a crime while under the influence of alcohol or drugs, are monitored for compliance with their conditions of probation, especially with an abstinence from the consumption of alcohol, during their probation period.

For example, ankle monitors (ankle bracelets or ankle shackles), which are attached to an ankle of the convicts, are used within the framework of this monitoring. The ankle monitor has a sensor, which can transdermally measure an alcohol concentration of the convicts. In the process, the sensor detects the alcohol diffusing through the skin to the ankle of the convicts.

The transdermally detected measured values of the alcohol concentration are then read out in a monitoring station or transmitted wirelessly to the monitoring station. The data collected in the monitoring station, a kind of data collection station, can be utilized by government agencies or probation officers for monitoring the conditions of probation.

The technical embodiment of a monitoring by means of an ankle monitor is subject to a plurality of legal requirements. Thus, for example, no gap may occur in the monitoring of the convicts, in which the convicts can obtain alcohol for themselves in an unnoticed manner. The technical embodiment of the ankle monitor itself is also subject to additional requirements of the user of the ankle monitor, for example, safety, wear comfort, operating comfort and no or only minimal visibility for third parties.

### SUMMARY

In addition to the use of such an ankle monitor or of such an electronic measuring device in case of convicts, additional areas of application of such systems are also possible. Consequently, the system for the transdermal monitoring of an alcohol concentration in a test subject according to the present invention presented below is not limited to the exemplary application for convicts described in the introduction, but also extends to additional application possibilities, so that a test subject is discussed below with reference to the system according to the present invention.

An object of the present invention is hence to provide an improved system for the transdermal monitoring of an alcohol concentration in a test subject, which meets legal requirements and any requirements provided by the client for the test subject with the simplest means possible and in a manner as comprehensive as possible.

The above object is accomplished by a system for the transdermal monitoring of a concentration of alcohol in a test subject having the features of the invention. Additional features and details of the present invention appear from the description and from the drawings.

The object is therefore accomplished by a system for the transdermal monitoring of an alcohol concentration in a test subject, wherein the system has: An electronic measuring device which is portable on the body of the test subject with a measuring module for the transdermal detection of measured values of the alcohol concentration in the test subject and with a transmission module for the transmission of the measured values to a relay station, the relay station with a receiving module for receiving measured values from the transmission module and with a sending module for sending the received measured values to a remotely located monitoring station, and at least one energy storage device, wherein an energy supply of the measuring module by means of the at least one energy storage device is uncoupled from the energy supply of the relay station by means of the at least one energy storage device.

The fact that the energy supply of the measuring module by means of the at least one energy storage device is uncoupled from the energy supply of the relay station by means of the at least one energy storage device means that the energy supply of the measuring module is detached from an energy supply of the relay station. As a result, for example, only the measuring module can be supplied with energy or only the relay station can be supplied with energy, without the respective other unit also having to be supplied with energy by the at least one energy storage device. This makes it possible that the measuring module can operate separately from the relay station.

The measuring module may especially comprise a sensor for the transdermal measurement of the measured values of the alcohol concentration in a test subject or, in other words, measurement of the transdermal concentrations as measured values. The sensor may especially be a gas sensor, which measures the concentration of the alcohol in the air on the skin of the test subject and/or in the sweat on the skin of the test subject. In this case, the alcohol diffuses through the skin of the test subject. Furthermore, the measuring module may also have a pump. This pump may be set up such that the pump feeds the air above the skin to the gas sensor.

The measuring module can further be set up such that the measured values contain the measuring times of the measured transdermal alcohol concentration. As a result, a measuring time may be associated with each measured transdermal alcohol concentration. The measuring times may be determined at defined intervals to one another or at defined times.

The at least one energy storage device may especially be a battery, for example, a lithium-ion-based battery.

Provisions may be made for the measuring device to have a memory for storing the measured values and for the measuring device to be set up with a measuring function to detect the measured values at different measuring times of a measuring time period and to store them as measured values in the memory.

As a result, the measured values at the different measuring times are stored in an aggregated or bundled manner in the memory. Thus, the measured values do not have to be transmitted to the relay station at each measuring time. Hence, the relay station does not have to permanently be in communication connection with the measuring device. The memory ensures that the measured values cannot be lost, even if, for example, an energy supply of the measuring device should be interrupted. The memory may, in addition, be coded such that a manipulation from outside is not possible.

The relay station may also otherwise have a memory, in which the measured values, which have been transmitted in an aggregated or bundled manner to the receiving module, are stored. Hence, the relay station also does not have to be in permanent contact with the monitoring station.

The measured values may each be stored in a legally compliant manner in the memory or in the memories. The measured values may, in addition, be reliably protected against external access. To this end, the measured values may be encrypted, so that they cannot be manipulated either during the transmission or in the memory. The corresponding key for decrypting the measured values may only be known to the monitoring station.

Provisions may be made in this case for the transmission module to be set up with a transmission function to transmit to the relay station in a bundled manner the measured values that have been measured at the different measuring times in the measuring time period and have been stored in the memory, at a transmission time, which is outside the measuring time period.

Accordingly, the transmission module with the transmission function has a function, with which measured values are first collected in a bundled manner in a measuring time period and are then transmitted in a bundled manner to the relay station. Compared with an individual transmission of each of the measured values to the relay station, this has the advantage that the measuring device can be operated in a substantially more energy-saving manner. Finally, the transmission of each of the measured values individually is more energy-intensive than a single transmission of bundled measured values.

In this case, it is sufficient in the application area of the monitoring of convicts for abstinence from alcohol that the values are not currently being transmitted during each measurement. It may, for example, already be sufficient when the measured values are transmitted only once per day to the relay station and are sent to the monitoring station from there. Accordingly, it is also not necessary, thanks to the uncoupling of the energy supply of the measuring module and the relay station, for the relay station to always be supplied with energy and to be available.

Provisions may be made for the transmission module and/or for the receiving module to be set up to be deactivated during the measuring time periods or to be in an energy-saving mode and to be activated at the transmission time or to end the energy-saving mode.

For example, the transmission module is able to trigger the activation of the receiving module or the ending of the energy-saving mode of the receiving module by means of the transmission or by means of a signal to the receiving module. In any case, energy available in limited quantities can thus be further saved. This is important since the measuring device should be configured as compact as possible, i.e., should have only a small energy storage device, so as to be invisible to third parties, if possible. The relay station should also have a compact configuration.

Provisions may further be made for the measuring device to have a first energy storage device for the energy supply of the measuring device and for the relay station to have a second energy storage device for the energy supply of the relay station. Consequently, an especially effective uncoupling of the energy supply of the measuring module from the energy supply of the relay station is achieved. The first energy storage device and the second energy storage device may be mobile energy storage devices here. The available energy is limited in case of such mobile energy storage devices. One of the two energy storage devices or both energy storage devices may be removable or replaceable from the measuring device and from the relay station or else be permanently installed. When the energy storage devices are permanently installed and are not replaceable, this has the advantage that the capacity of the energy storage devices in case of identical compact size may turn out to be higher.

Provisions may further be made for the measuring device to be portable on the leg, especially on the ankle, of the test subject. Provisions may especially be made for the measuring device to be configured as an ankle monitor for the ankle of the test subject.

Even though the measuring device may theoretically also be used on other body parts, especially on extremities, of the test subject, i.e., for example, on an arm or wrist of the test subject, the visibility there is increased compared to an attachment on the leg. Further, there is the risk that the operability of the measuring device will be impaired. This may be the case, for example, in case of a contact of the measuring device with water. In particular, this is more rarely the case on the ankle of the test subject than on the wrist in a normal everyday life.

The ankle monitor may have, for example, a strap or belt or the like, by means of which the ankle monitor can be put on the ankle of the test subject. Provisions may be made for the ankle monitor to be able to be put on the ankle of the test subject such that it can only be removed by a person who is authorized, for example, by a probation officer, at least without having signs of violence being used. Thus, the ankle monitor may have, for example, a lock, for example, an electronic lock, which can only be opened with a corresponding electronic key.

Provisions may also be made for the measuring device to be configured as a measuring apparatus and for the relay station to be a relay device configured separately from the measuring apparatus.

In other words, the measuring apparatus and the relay device have their own housings, which are separate from one another. Due to the transfer of the relay station into a relay device configured separately from the measuring apparatus, it is made possible that the measuring device or the measuring apparatus, which is portable on the body of the test subject, may have an especially compact configuration. The relay device may likewise have a compact configuration. Further, it is, as a result, not absolutely necessary for the test subject to have to carry the relay device with the test subject constantly.

Provisions may be made in this case for the relay device to be portable on the body, especially on the arm, more specifically on the wrist, of the test subject.

Provisions may be made in this case for the relay device to be configured as a wristband and/or as a watch for the wrist of the test subject.

Consequently, the relay device no longer has to remain invisible to third parties, but rather may be worn inconspicuously as a fashion accessory. The watch has at least one time display and thus it distracts from the fact that it is actually primarily a relay device in a system for monitoring the transdermal alcohol concentration in a test subject. In this case, it may, furthermore, be a smartwatch. This smartwatch may have additional functionalities, for example, fitness tracking, phone functions, etc.

In this connection, provisions may further be made for the transmission module and the receiving module to be set up for the wireless transmission and receiving of the measured values by means of near field communication or short-range radio technology.

Especially a wireless personal area network (WPAN), for example, Bluetooth® (Bluetooth® is a registered trademark of Bluetooth SIG, Inc.), may be used as short-range radio technology. A short-range radio technology may especially be used with low energy demand, for example, with Bluetooth Low Energy.

A transmission standard based on RFID technology is meant as near field communication. The memory with the measured values can be read out by means of the near field communication in an especially energy-saving manner. To this end, the relay device only has to be brought into the near field of the transmission module. The relay device may then automatically carry out the sending of the measured values to the remotely located monitoring station. The relay device can prompt the test subject at the transmission time, for example, to transmit to the relay device the measured values recorded in the memory by means of arranging the relay device in the near field of the transmission module.

Provisions may further be made for the transmission module and the receiving module to have each a transmission connection for an additional wired transmission and receiving of measured values.

This may be used as a backup solution if the wireless communication between the transmission module and the receiving module fails. This may be displayed to the test subject, for example, by an indication on a relay device, which is configured as a smartwatch. The test subject may then be prompted to carry out a wired transmission. Thus, the test subject can be prevented from not carrying out a transmission because of a technical defect, i.e., for example, a convict can be prevented from violating his conditions of probation because of an incomplete transmission of measured values.

Provisions may further be made for the relay device to be configured solely for communication with the measuring apparatus and with the monitoring station.

The relay device may be set up only for communication with the measuring apparatus and with the monitoring station especially through defined communication channels, proprietary ports, an encryption of the communication or data or the like. Thus, for example, no computer may be coupled with the relay device or may be brought at least in communication with this relay device since the relay device is encrypted and the computer does not have the corresponding software for the decryption of the data located on it or a corresponding port. Consequently or especially, the relay device may not be a smartphone, a tablet, a computer or the like, especially not from a manufacturer other than the manufacturer of the measuring apparatus. When the relay device is set up only for communication with the measuring apparatus and with the monitoring station, manipulation attempts of the relay device and thus of the recorded measured values are avoided or at least reduced.

Furthermore, provisions may be made for the sending module to have a cell phone interface for sending the received measured values to the monitoring station.

This allows the test subject a great freedom of movement when carrying along the relay device. Thus, the relay device can send to the monitoring station the measured values, for example, at the request of the monitoring station, by means of the cell phone interface practically at any time, as long as there is cell phone reception. The cell phone interface may be any desired cell phone standard (e.g., GSM, LTE, 5G). Further, the relay station may additionally have, as a backup solution or as an alternative, a wired interface into the internet, for example, by means of a LAN connection with an internet-enabled modem.

In addition, provisions may be made for the system to have a mobile additional energy storage device for charging the at least one energy storage device.

The safety of the system is increased by means of the additional energy storage device since the measuring device does not have to be charged directly at a socket. This could lead to an electric shock of the test subject in case of a technical defect, which can be avoided by means of the mobile additional energy storage device.

The mobile additional energy storage device has, consequently, an energy storage device itself. The energy storage device of the mobile additional energy storage device may, in turn, be safely charged at the power plug or a socket, since the test subject does not wear the mobile additional energy storage device on his body.

The present invention also especially comprises a system for monitoring a transdermal alcohol concentration in a test subject, wherein the system has:
(a) an electronic measuring device, which is portable on the body of the test subject, with a measuring module for the detection of measured values of the transdermal alcohol concentration in the test subject and with a transmission module for the transmission of the measured values to a relay station,
(b) the relay station with a receiving module for receiving the measured values from the transmission module and with a sending module for sending the received measured values to a remotely located monitoring station,
(c) an energy storage device for the energy supply of the measuring module, and
(d) a mobile additional energy storage device for charging the at least one energy storage device.

In this case, the above-mentioned system may further have the features of the present invention mentioned above and/or below, so that the contents of the patent application also extend to the above-mentioned system. In this case, the at least one energy storage device can be replaced in the features with the energy storage device, since the above-mentioned system does not provide any uncoupling of the energy supply of the measuring module from the energy supply of the relay station, but solely an energy storage device for the energy supply of the measuring module.

In this case, provisions may be made for the at least one energy storage device to be set up for wireless energy reception from the mobile additional energy storage device and for the at least one mobile additional energy storage device to be set up for wireless energy transmission to the at least one energy storage device.

Consequently, the measuring device may have a wireless energy receiving device. The mobile additional energy storage device may have a wireless energy transmitting device. The wireless energy receiving device and the wireless energy transmitting device may be set up, for example, for inductive charging.

Provisions may also be made for the at least one energy storage device and for the mobile additional energy storage device to be coded for each other such that the at least one energy storage device may only be charged by means of the mobile additional energy storage device.

The coding may be carried out, for example, in case of a wireless energy transmission such that the mobile additional energy storage device or the at least one energy storage device or the measuring device is identified with each other by a code or key, for example, a handshake can be carried out. In case of a wired energy transmission, the coding can take place by the selection of proprietary plugs and outlets.

Provisions may therefore be made for the measuring device and/or for the mobile additional energy storage device to have a proprietary outlet for an energy storage device charging cable to connect the measuring device to the mobile additional energy storage device.

Instead of the designation proprietary, the designation non-standardized may also be used. Thus, in any case it is at least one manufacturer-specific, especially system-specific, outlet, which can be used only for the measuring device or for the mobile additional energy storage device. It is avoided as a result that the measuring device can be charged by means of a commercially available and/or standardized charging cable. This rules out attempts at manipulation and unsafe charging cables, by means of which the test subject could be put at risk, for example, if the test subject would charge the measuring device directly at the socket instead of by means of the mobile additional energy storage device.

In this case, provisions may be made for the system to have the energy storage device charging cable for the wired transmission of energy from the mobile additional energy storage device to the at least one energy storage device, wherein the energy storage device charging cable has a proprietary plug for the proprietary outlet.

Consequently, the energy storage device charging cable is provided with the proprietary plug, so that it cannot be used for other purposes and cannot be manipulated or damaged.

Furthermore, provisions may be made for the mobile additional energy storage device to have an energy supply charging cable for charging the mobile additional energy storage device at a socket, wherein the energy supply charging cable is connected inseparably to the mobile additional energy storage device or has a proprietary outlet on a connection side to the mobile additional energy storage device.

Also on the charging side of the mobile additional energy storage device to the socket, it can be consequently guaranteed that no third-party cables are used for charging and thus no undesired influences are exerted.

Furthermore, provisions may be made for the mobile additional energy storage device to have a securing mechanism which is set up such that the mobile additional energy storage device cannot simultaneously charge the at least one energy storage device and be charged itself.

The securing mechanism thus guarantees that the test subject cannot get an electric shock from a socket, at which the mobile additional energy storage device is charged, if one of the components should have a technical defect or is subject to mishandling. The safety of the test subject during the charging of the measuring device may, as a result, be increased in a simple and yet effective manner.

Additional actions improving the present invention appear from the following description about an exemplary embodiment of the present invention, which is shown in the figures. All features and/or advantages appearing from the claims, from the description or from the drawings, including design details and arrangements in space, may be essential to the present invention both by themselves and in the various combinations.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a side perspective view of a test subject with a system according to an exemplary embodiment of the present invention;

FIG. 2 is a schematic view of the system according to FIG. 1;

FIG. 3 is another schematic view of a part of the system according to FIG. 1;

FIG. 4 is a schematic diagram of a charging in a system according to FIG. 1; and

FIG. 5 is a schematic diagram of a defection of measured values and a transmission of measured values in a system according to FIG. 1.

### DESCRIPTION OF PREFERRED EMBODIMENTS

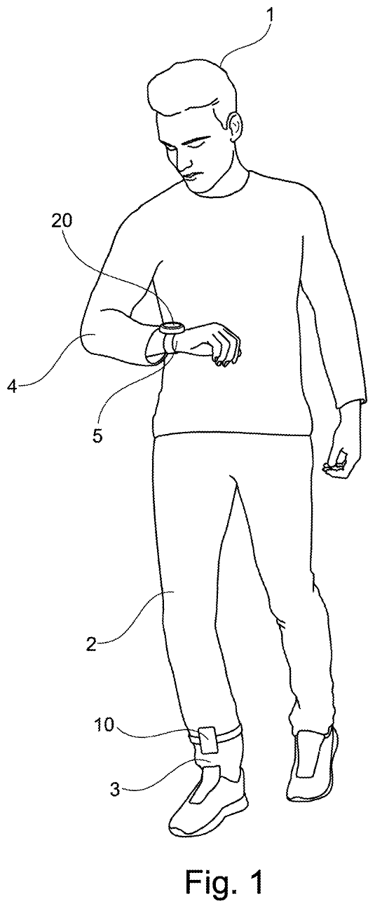

Referring to the drawings, elements with identical function and mode of operation are all provided with the same reference numbers in FIGS. 1 through 5.

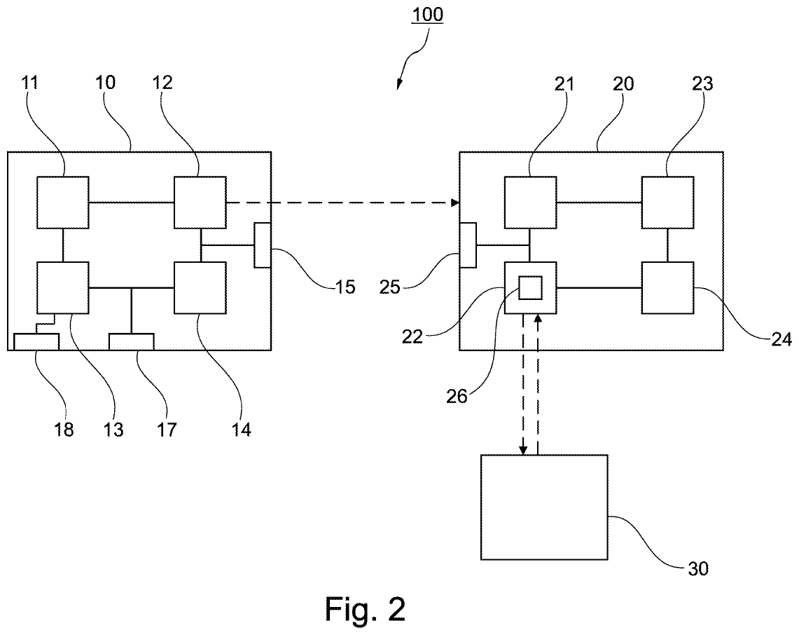

The test subject 1 in FIG. 1 is equipped with a measuring device 10 and with a relay station 20 of the system 100 (see FIG. 2). In the present exemplary embodiment, the measuring device 10 and the relay station 20 are configured as devices formed separately from one another and are therefore designated below as the measuring device 10 and the relay device 20.

The measuring device 10 is configured as an ankle monitor (ankle bracelet), which in the present case is put on a leg 2, especially on an ankle 3, of the test subject 1. The measuring device 10 is located under the trousers of the test subject 1, so that the measuring device 10 is not visible from outside.

The relay device 20 is configured as a smartwatch, which in the present case is put on an arm 4, especially on a wrist 5, of the test subject 1. In FIG. 1, the test subject 1 is looking at the relay device 20, which displays the current time to the test subject 1. Furthermore, the relay device can be equipped with a GPS function, which makes it possible to follow his current location. This may be helpful when the test subject 1 is a convict who is on probation and is being monitored. As an alternative or in addition, the measuring device 10 may be equipped with such a GPS function as well.

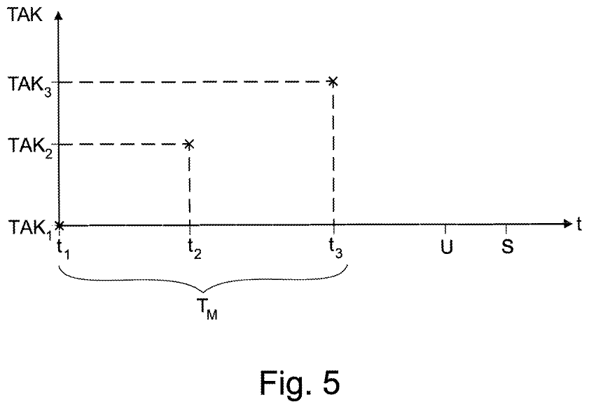

The measuring device 10 is secured to the test subject 1 and cannot be independently removed by the test subject 1, so that the measuring device 10 can continuously detect measured values of the transdermal alcohol concentration TAK (see FIG. 5). The relay device 20 may, by contrast, be removed independently by the test subject 1.

FIG. 2 shows in a schematic view the system 100 from FIG. 1 with the measuring device 10 and with the relay device 20. The measuring device 10 comprises a measuring module 11, which in turn has a gas sensor, not shown, for the measurement of measured values of the transdermal alcohol concentration TAK at the skin of the test subject 1. Furthermore, the measuring device 10 comprises a transmission module 12 for transmitting the transdermal alcohol concentration TAK measured values measured by the measuring module 11.

Furthermore, the measuring device 10 has an energy storage device 13 for the energy supply of the measuring module 11 and of the transmission module 12. Further, the measuring device 10 has a memory, in which the measured values of the measuring module 11 can be stored. Finally, the measuring device 10 has a transmission connection 15, a wireless energy receiving device 17 and a proprietary outlet 18.

The transmission module 12 transmits the measured values from the memory 14 to a receiving module 21 of the relay device 20 in a bundled manner. The relay device 20 likewise has, in turn, a memory, in which the received measured values can be stored. An additional energy storage device 23 in the relay device 20 supplies the receiving module 21 and a sending module 22 with energy. The relay device 20 also has a transmission connection 25.

Provisions may further be made that the energy storage device 23 can be charged in the relay device 20 with energy by cable and/or wirelessly. Since the relay device 20 can be removed by the test subject 1, provisions may be made for the relay device 20 to be charged directly at an outlet.

By means of the transmission connections 15, 25, a wired transmission of the measured values can be carried out from the measuring device 10 to the relay device 20 if a wireless transmission by means of the transmission module 12 and the receiving module 21, for example, by means of near field communication or short-range radio technology, should fail.

The sending module 22 has, furthermore, a cell phone interface 26. It is possible by means of the cell phone interface 26 to transmit the measured values received from the measuring device 10 and possibly temporarily stored in the memory 24 to a remotely located monitoring station, where the measured values will be processed.

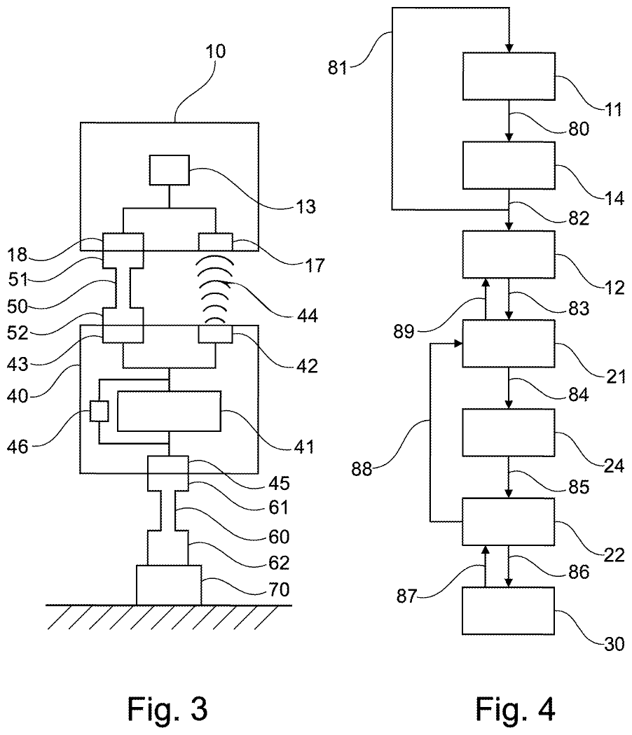

FIG. 3 schematically shows the procedure during the charging of the energy storage device 13 of the measuring device 10, which is worn on the ankle 3 of the test subject 1. For this purpose, the system 100 further comprises a mobile additional energy storage device 40. The measuring device 10 is shown here in a simplified manner only with the components that are relevant for the charging.

The mobile additional energy storage device 40 has, in turn, an energy storage device 41. The energy storage device 41 in the mobile additional energy storage device 40 or the mobile additional energy storage device 40 is first charged at a socket 70. For this purpose, the system 100 further has an energy supply charging cable 60.

The energy supply charging cable 60 has a standardized plug 62, which can be inserted into the socket 70 or into a corresponding power plug, which is not shown and can be inserted at the socket 70. The energy supply charging cable 60 also has a proprietary plug 61 for a proprietary outlet 45 of the mobile additional energy storage device 40. When the energy supply charging cable 60 is connected to the household socket 70 and the energy supply charging cable 60 is connected to the mobile additional energy storage device 40, the energy storage device 41, of the mobile additional energy storage device 40, is charged.

This procedure of charging at the socket 70 is, however, prevented by means of a securing mechanism 46 in the mobile additional energy storage device 40 when the measuring device 10 is charged by means of an energy storage device charging cable 50 at the same time.

The energy storage device charging cable 50 is connected to a proprietary outlet 18 of the measuring device 10, on the one hand, and to a proprietary outlet 43 of the mobile additional energy storage device 40, on the other hand. To this end, the energy storage device charging cable 50 has proprietary plugs 51, 52 at each end.

As an alternative or in addition to this, the measuring device 10 has a wireless energy receiving device 17. The mobile additional energy storage device 40 has, in turn, a wireless energy transmitting device 42, which, just as the proprietary outlet 43, is, in turn, connected to the energy storage device 41. Thus, the energy storage device 13 of the measuring device 10 can also be charged by wireless energy transmission 44, which ensures a greater safety and a higher comfort during the charging of the measuring device 10, which cannot be removed by the test subject 1. In this respect, the wired charging by means of the energy storage device charging cable 50 represents a fallback solution or an alternative solution.

FIG. 4 schematically shows the procedure in case of the detection, storage, transmission and sending of the measured values. For this purpose, various steps are provided, which are each characterized by the procedures 80 through 89, which connect each of the components involved in the process.

A measured value is first detected by means of the measuring module 11. This measured value is according to procedure 80 transmitted to the memory 14, where the measured value is stored. According to procedure 81, the measured value is then again transferred to the measuring module 11, which carries out a new measurement at a defined time interval and also transmits the additional measured value to the memory 14.

At a defined time or at the request of the monitoring station 30, the measured values are provided from the memory 14 to the transmission module 12 according to the procedures 87, 88, 89. The transmission module 12 transmits the bundled measured values according to procedure 83 to the receiving module 21 of the relay device 20.

Having been received at the receiving module 21 the measuring values can be temporarily stored in the memory 24 of the relay device 20 according to procedure 84. According to procedure 85, they are then provided to the sending module 22 and are finally sent to the monitoring station 30 according to procedure 86.

FIG. 5 schematically shows a detection of the measured values as well as the transmission and the sending of the measured values detected.

In a measuring time period T_{M}, measurements are carried out by the measuring module 11 of the measuring device at different measurement times, at the three measurement times t₁, t₂, t₃ in the present case. The measured values are formed by the transdermal alcohol concentrations TAK₁, TAK₂, TAK₃ detected at the measurement times t₁, t₂, t₃.

After the measuring time period T_{M}, the detected measured values, which have been stored in the memory 14, are transmitted from the transmission module 12 to the receiving module 21 at a transmission time U. After the transmission time U, the measured values, which have been received by the receiving module 21 and have possibly been temporarily stored in the memory 24, are sent to the transmission station 30 at the sending time S.

While specific embodiments of the invention have been shown and described in detail to illustrate the application of the principles of the invention, it will be understood that the invention may be embodied otherwise without departing from such principles.

### LIST OF REFERENCE CHARACTERS

1 Test subject
2 Leg
3 Ankle
4 Arm
5 Wrist
10 Measuring device, measuring apparatus
11 Measuring module
12 Transmission module
13 Energy storage device
14 Memory
15 Transmission connection
17 Wireless energy receiving device
18 Proprietary outlet
20 Relay station, relay device
21 Receiving module
22 Transmission module
23 Energy storage device
24 Memory
25 Transmission connection
26 Cell phone interface
30 Monitoring station
40 Mobile additional energy storage device
41 Energy storage device
42 Wireless energy transmitting device
43 Proprietary outlet
44 Wireless energy transmission
45 Proprietary outlet
50 Energy storage device charging cable
51 Proprietary plug
52 Proprietary plug
60 Energy supply charging cable
61 Proprietary plug
62 Standardized plug
70 Socket
80-89 Procedure
TAK Transdermal alcohol concentration
T_{M} Measuring time period
T Measuring time
U Transmission time
S Sending time

What is claimed is:

1. A system for transdermal monitoring of an alcohol concentration in a test subject, the system comprising:
an electronic measuring device which is portable on a body of the test subject, the electronic measuring device comprising:
a measuring module for the transdermal detection of measured values of the alcohol concentration in the test subject; and
a transmission module for transmission of the measured values;
a relay station, the relay station comprising:
a receiving module for receiving measured values, wherein the measured values are transmitted by the transmission module to the relay station and the measured values are received at the receiving module from the transmission module; and
a sending module for sending the received measured values to a remotely located monitoring station; and
at least one energy storage device providing an energy supply of the measuring module, wherein the energy supply of the measuring module is uncoupled from an energy supply of the relay station by means of the at least one energy storage device.

2. A system in accordance with claim 1, wherein:
the electronic measuring device further comprises a memory for storing the measured values;
the electronic measuring device is configured with a measuring function to detect the measured values at different measuring times of a measuring time period and to store the measured values at different measuring times as measured values in the memory.

3. A system in accordance with claim 2, wherein the transmission module is configured with a transmission function to transmit the measured values as a bundled transmission to the relay station, which measured values are measured at different measuring times in the measuring time period and are stored in the memory, at a transmission time which is outside the measuring time period.

4. A system in accordance with claim 3, wherein at least one of the transmission module and the receiving module is configured to be deactivated during the measuring time period or to be in an energy-saving mode and to be activated at the transmission time or to end the energy-saving mode.

5. A system in accordance with claim 1, wherein:
the at least one energy storage device providing the energy supply of the measuring module is a first energy storage device; and
the relay station has a second energy storage device for the energy supply of the relay station.

6. A system in accordance with claim 1, wherein the electronic measuring device is connected to the leg of the test subject so as to be portable on the leg.

7. A system in accordance with claim 1, wherein:
the electronic measuring device is configured as a measuring apparatus; and
the relay station comprises a relay device configured separately from the measuring apparatus.

8. A system in accordance with claim 7, wherein the relay device is connected to the arm of the test subject so as to be is portable on the arm of the test subject.

9. A system in accordance with claim 8, wherein the relay device is configured as a wristband for the wrist of the test subject or as a watch for the wrist of the test subject or as both a wristband for the wrist of the test subject or as a watch for the wrist of the test subject.

10. A system in accordance with claim 7, wherein the transmission module and the receiving module are configured for wireless transmission and receiving of the measured values by means of near field communication or short-range radio technology.

11. A system in accordance with claim 10, wherein the transmission module and the receiving module further have each a transmission connection for an additional wired transmission and receiving of the measured values.

12. A system in accordance with claim 7, wherein the relay device is configured solely for communication with the measuring apparatus and with the monitoring station.

13. A system in accordance with claim 1, wherein the sending module comprises a cell phone interface for sending the received measured values to the monitoring station.

14. A system in accordance with claim 1, further comprising a mobile additional energy storage device for charging the at least one energy storage device.

15. A system in accordance with claim 14, wherein the at least one energy storage device is configured for wireless energy reception from the mobile additional energy storage device and the at least one mobile additional energy storage device is configured for wireless energy transmission to the at least one energy storage device.

16. A system in accordance with claim 14, wherein the at least one energy storage device and the mobile additional energy storage device are coded for each other such that the at least one energy storage device may only be charged by means of the mobile additional energy storage device.

17. A system in accordance with claim 14, wherein at least one of the electronic measuring device and the mobile additional energy storage device has a proprietary outlet for an energy storage device charging cable for connecting the electronic measuring device to the mobile additional energy storage device.

18. A system in accordance with claim 17, further comprising an energy storage device charging cable for the wired transmission of energy from the mobile additional energy storage device to the at least one energy storage device, wherein the energy storage device charging cable has a proprietary plug for the proprietary outlet.

19. A system in accordance with claim 14, wherein:
the mobile additional energy storage device comprises an energy supply charging cable for charging the mobile additional energy storage device at a socket; and
the energy supply charging cable is connected inseparably to the mobile additional energy storage device or has a proprietary outlet on a connection side to the mobile additional energy storage device.

20. A system in accordance with claim 14, wherein the mobile additional energy storage comprises a securing mechanism configured to prevent the mobile additional energy storage device from simultaneously charging the at least one energy storage device and being charged itself.
